SEQUENCE LISTING (2 sequences)

SEQ ID NO: 1 - DNA, 30 bp, Artificial Sequence
  capture oligomer sequence
atcttcatgttggagctttttatcgtaaag

SEQ ID NO: 2 - DNA, 30 bp, Artificial Sequence
  signal oligomer sequence
ttgtattgctattatcgtcaacgattgtga

(19) United States
(12) Patent Application Publication
(10) Pub. No.: US 2020/0123595 A1
(43) Pub. Date: Apr. 23, 2020

(54) Method for Detecting Bacteria

(71) Applicant: GFC Diagnostics Ltd., Northamptonshire (GB)

(72) Inventor: Graham Mock, Northamptonshire (GB)

(21) Appl. No.: 16/332,672

(22) PCT Filed: Sep. 11, 2017
(86) PCT No.: PCT/EP2017/072785

§ 371 Date: Mar. 12, 2019

(30) Foreign Application Priority Data
Sep. 12, 2016 (GB) 1615466.8

(51) Int. Cl. C12Q 1/689; C12Q 1/6841; C12M 1/34

(52) U.S. Cl. CPC C12Q 1/689; C12M 41/46; C12Q 1/6841

(57) ABSTRACT

The present proposals relate to methods for detection of whole-cell bacteria, for example whole-cell pathogenic bacteria, in particular drug-resistant bacteria. These proposals also include apparatus and kits for use in such methods. Described is a method of detecting specific whole-cell bacteria, the method comprising: adding a probe composition to a sample comprising whole bacterial cells, the probe composition comprising a capture probe L-O1 and a signal probe X-=2, wherein L is at least one moiety that is one half of a binding pair, X is at least one signal moiety that provides an observable signal, and each O1 and O2 is independently a 15-150 nucleotide oligomer that is complementary to a portion of a nucleotide sequence of the specific bacteria; bringing the sample/probe composition mixture into contact with a substrate on which a moiety L′ is immobilised wherein L′ is complementary to and binds with the moiety L; washing the substrate to remove excess sample and probe composition; and detecting the observable signal.

### CROSS REFERENCE TO RELATED APPLICATIONS

This application is a § 371 National State Application of PCT/EP2017/072785 filed Sep. 11, 2017, which claims priority to GB 1615466.8 filed Sep. 12, 2016.

### SEQUENCE LISTING

The instant application contains a Sequence Listing which has been submitted in ASCII format via EFS-Web and is hereby incorporated by reference in its entirety. The ASCII copy of the Sequence Listing, which was created on Sep. 12, 2016, is named Mock_Sequence_Listing.txt and is 621 bytes in size.

### TECHNICAL FIELD

The present proposals relate to methods for detection of whole-cell bacteria, for example whole-cell pathogenic bacteria. These proposals also include apparatus and kits for use in such methods.

### BACKGROUND

Detection of bacteria, in particular pathogenic bacteria, is important in a wide range of areas such as healthcare, food hygiene, and security. It is especially important to detect bacteria quickly and accurately and to minimise risk to people undertaking the analysis.

In particular, detection of so-called “superbugs” such as methicillin-resistant Staphylococcus aureus (MRSA) and Clostridium difficile (C. diff) is important in healthcare settings and current slow sample analysis presents significant problems in development of a point-of-care test for these pathogens.

Existing methods of bacterial detection typically rely on polymerase chain reaction (PCR) techniques to identify the bacteria in a sample. While PCR techniques are accurate, they require significant sample preparation to extract suitable DNA for analysis and they are typically time-consuming and costly. These techniques also require specialist training both in terms of undertaking the analysis and interpreting the results. Even more recent advances such as “real-time PCR” still take several hours to reach a result and have the same sample preparation and specialist equipment requirements.

Electrochemical analysis techniques have also been used in bacterial detection but in some cases accuracy has been problematic and impurities in samples can impact results so these techniques may be harder to use with “raw” samples, i.e. with no pre-processing prior to analysis.

General immunosorbent assay techniques are known which use antibody binding to detect the presence of a substance, for example in enzyme-linked immunosorbent assay (ELISA) techniques and “sandwich” ELISA techniques. Also known are reagents that can be used in so-called enzyme-linked oligosorbent assay (ELOSA) methods. These compounds use oligomeric probes crosslinked to capture moieties and signal moieties in which the oligomers are designed to hybridise to a target sequence of interest by complementary base-pairing. Such reagents are described, for example, in WO 2007/132207.

There is still a need for a rapid, safe method for detecting whole-cell bacteria, particularly one that requires little or no specialist training and uses low cost materials, and apparatus for conducting such a method.

### SUMMARY

The present proposals provide a method of detecting specific whole-cell bacteria, the method comprising:

adding a probe composition to a sample comprising whole bacterial cells, the probe composition comprising a capture probe L-O1 and a signal probe X-O2, wherein L is at least one moiety that is one half of a binding pair, X is at least one signal moiety that provides an observable signal, and each O1 and O2 is independently a 15-150 nucleotide oligomer that is complementary to a portion of a nucleotide sequence of the specific bacteria;

bringing the sample/probe composition mixture into contact with a substrate on which a moiety L′ is immobilised wherein L′ is complementary to and binds with the moiety L;

washing the substrate to remove excess sample and probe composition; and

detecting the observable signal.

These proposed methods may, in some cases, provide detection results after only a short incubation time (the time for which the sample is in contact with the probe composition prior to the washing step). For example, in some cases an incubation step of less than 30 minutes is used.

These proposals also provide a kit for the detection of specific whole-cell bacteria, the kit comprising:

a probe composition comprising a capture probe L-O1 and a signal probe X-O2, wherein L is at least one moiety that is one half of a binding pair, X is at least one signal moiety that provides an observable signal, and each O1 and O2 is independently a 15-150 nucleotide oligomer that is complementary to a portion of a nucleotide sequence of the specific bacteria; and

an assay device comprising: a body including an assay chamber containing a substrate on which a moiety L′ is immobilised wherein L′ is complementary to and binds with the moiety L; a sample collector/dispenser for collecting a sample to be assayed and dispensing a quantity of it into the assay chamber, wherein the body and the collector/dispenser are engageable together.

The assay device itself also forms part of the present proposals when it contains the capture probe, and signal probe along with the substrate on which the moiety L′ is immobilised.

Use of an assay device as defined herein in a method of detecting specific whole-cell bacteria is also part of the present proposals.

### BRIEF DESCRIPTION OF THE FIGURES

FIG. 1a-c shows a generalised scheme for completion of an assay of the present invention with FIG. 1a showing the target loading, FIG. 1b showing washing, and FIG. 1c showing colour development stages.

FIGS. 2 and 3 show a comparison of the negative and positive data from Example 1. FIG. 3 is an enlargement of a portion of FIG. 2.

FIG. 4 shows a plot of absorbance of the MRSA and MSSA samples in Example 1 at various selected time points.

### FURTHER DEFINITIONS; OPTIONS AND PREFERENCES

The present proposals provide methods and apparatus for detection of specific whole-cell bacteria. These methods do not require significant sample processing prior to analysis. For example, in preferred embodiments no cell lysis step is required or any other DNA extraction technique. The sample does need to be in liquid form for the analysis steps so if the raw sample is a solid or gel, the present methods may include addition of a suitable solvent to present the sample in liquid form. For example, if the sample is taken as a swab from a human or animal or from a surface, the sample may be extracted from the swab with a solvent. In preferred cases a suitable solvent is water. However, in some embodiments, samples can be used directly as obtained, e.g. unprocessed body fluid samples such as blood, saliva, sputum, sweat etc. In preferred embodiments, the present methods do not include a DNA extraction step. In some embodiments, the methods do not include a DNA extraction step prior to addition of the probe composition. The present methods do not include a polymerase chain reaction (PCR) step.

The present proposals provide significant advantage due to this ability to detect specific bacteria in whole-cell samples. This avoids the need for sample processing steps that may involve hazardous chemicals or require specialised techniques or equipment. Therefore the present methods are typically simpler and cheaper than known techniques.

The present methods may, in some cases, provide detection results after only a short incubation time (the time for which the sample is in contact with the probe composition prior to the washing step). The required incubation time may depend on the nature of the specific bacteria being detected and on the nature of the signal probe and capture probe sequences. In preferred methods, the incubation time is less than 1 hour, preferably less than 45 minutes, preferably less than 30 minutes, preferably less than 15 minutes, for example less than 10 minutes. This short incubation time may, in some cases, be a result of the nature of the oligomers O1 and O2, e.g. oligomer length and/or binding specificity to the target bacteria sequence.

The present methods are suitable for detection of a wide range of different bacteria. For example, gram-positive and gram-negative bacteria may be detected. The methods are suitable for detection of bacterial pathogens such as agents that may be present in healthcare environments or agents that may be used as biological warfare agents. For example, the present methods may be useful to detect specific bacteria selected from: Bacillus anthracis (anthrax), Yersinia pestis (plague), methicillin-resistant Staphylococcus aureus (MRSA), methicillin-susceptible Staphylococcus aureus (MSSA), Clostridium difficile (C. diff), Vancomycin-resistant Enterococci (VRE), Carbapenemase-producing enterobacteriaceae (CPE), Multidrug resistant Mycobacterium tuberculosis (MDR-TB), colistin resistant bacteria, Extended-spectrum β-lactamase producing Gram-negative bacteria (ESBLs), Klebsiella pneumoniae carbapenemase (KPC) producing Gram-negative bacteria, Streptococcus pneumoniae, Haemophilus influenza, Mycobacterium tuberculosis, Shigellae (e.g. S. dysenteriae, S. flexneri, S. boydii, S. sonnei), Campylobacter, Salmonellae (e.g. Salmonella enterica, Salmonella bongori, Salmonella Typhi), Escherichia coli, Lysteria monocytogenes, Shewanella oneidensis, Neisseria meningitides, Treponema pallidum (syphilis), Neisseria gonorrhoeae (gonorrhoea), Chlamydia trachomatis (chlamydia), Porphyromonas gingivalis. Drug resistant bacteria provide a particularly preferred target for detection, for example MRSA, CPE, MDR-TB, and colistin resistant bacteria. In particular, the present methods have the ability to detect specific bacteria by appropriately selecting the nucleotide oligomers O1 and O2 to be complementary to the desired bacterial nucleotide sequence. Therefore these methods provide the ability to detect, for example, MRSA in the presence of other bacteria, e.g. MSSA. This means that the present methods can provide a definitive test for the presence of a specific bacteria (e.g. a bacteria selected from the list above) even in the presence of other bacteria. In preferred embodiments, the present methods are effective to detect MRSA or C. diff., in particular to detect MRSA as distinct from MSSA.

In some cases the methods and apparatus may be used to detect the presence of any one or more bacteria in a range of different specific possibilities. For example, the method may be suitable to detect any one of a plurality of different bacteria (e.g. bacteria selected from those presented herein). Such methods or apparatus may be used, for example, to detect whether any one of a selected range of bacteria is present in a sample. In a healthcare setting this could be used to determine the presence of any of, for example, MRSA, C. diff., or VRE in a sample.

The present proposals use a capture probe L-O1 and a signal probe X-O2 wherein L is at least one moiety that is one half of a binding pair L/L′, X is at least one signal moiety that provides an observable or detectable signal, and each O1 and O2 is independently a 15-150 nucleotide oligomer that is complementary to a portion of a nucleotide sequence of the specific bacteria.

Each of the oligomers O1 and O2 is independently selected to be complementary to a portion of the nucleotide sequence of the target specific bacteria. The length of each of O1 and O2 is 15-150 nucleotides. Above about 150 nucleotides in length, there is not much significant improvement in specificity of the binding to the selected bacteria but it may start to take longer for the oligomers to hybridise to the bacterial sequence and be difficult to get them to bind close together. Below about 15 nucleotides in length, the specificity of binding to the bacterial sequence is lowered with the risk of false positive results due to O1 and O2 hybridising to a bacteria other than the specific target. In preferred embodiments, the length of each of O1 and O2 is independently selected from a nucleotide number of: less than 150, less than 100, less than 75, less than 50, or less than 25. In preferred embodiments, the length of each of O1 and O2 is independently selected from a nucleotide number of: more than 15, more than 20, more than 30, more than 40, or more than 50. Any of these upper and lower limits for the length of O1 and O2 may be combined to for a range. For example, in preferred embodiments, the length of each of O1 and O2 is independently selected from a nucleotide number in the range: 15-150, 15-100, 20-100, 10-75, 10-50, 20-75, 20-50, or 30-50. Preferably the length of O1 and O2 is the same. Preferably the length of O1 and O2 is about 30 nucleotides.

The oligomers O1 and O2 are complementary to the nucleotide sequence of the specific target bacteria so that O1 and O2 hybridise to a desired target region of the bacterial DNA. In preferred embodiments of O1 and O2, their nucleotide sequence is 100% complementary to a section of the DNA nucleotide sequence of the target bacteria, for example at least 99% complementary, or at least 98%, or at least 95%, or at least 90% complementary.

The oligomers O1 and O2 are selected to bind close to each other on the target strand. In preferred aspects, O1 and O2 are selected to bind to sites separated by about 1-50 nucleotides, preferably 5-25, preferably 5-15, more preferably 5-10, e.g. about 9 nucleotides. This close proximity of the binding sites helps to minimise problems with the assay in terms of DNA breakage. For example, if the DNA strand breaks between O1 and O2, problems with false positives or false negatives in the detection could be experienced.

In some cases, the capture probe comprises a capture oligomer which is complementary to part of the MRSA DNA sequence, the capture probe having the sequence of SEQ ID NO: 1:

3′-gaa atg cta ttt ttc gag gtt gta ctt cta-5′

In some cases the signal probe comprises a signal oligomer which is complementary to part of the MRSA DNA sequence, the capture probe having the sequence of SEQ ID NO: 2:

3′-ag tgt tag caa ctg cta tta tcg tta tgt t-5′

These two specific probes O1 and O2 when used together bind to MRSA with a space of 9 nucleotides between them. These sequences provide particularly beneficial properties in terms of good recognition of MRSA while maintaining rapid binding and with a suitably small gap between the bound sequences to minimise false positive/negative results due to, for example, DNA breakage. Therefore, these two probes O1 and O2 are particularly preferred where MRSA is the target bacteria.

The capture probe L-O1 in these proposals includes the moiety L which is one half of a binding pair L/L′. The other half of the binding pair is immobilised on the substrate so that when L and L′ come into contact, the L-O1 unit (and any moiety to which it is bound) is immobilised on the substrate due to binding interaction between L and the complementary L′. This allows the target bacterial DNA to which O1 is bound to be immobilised on the substrate. The binding pairs L/L′ are not particularly limited and may be any known complementary binding pair. For example they may be biotin/streptavidin, biotin/avidin, fluorescein/anti-fluorescein, digoxigenin/anto-digoxigenin. Preferably the binding pair L/L′ is biotin/streptavidin. In preferred embodiments of this pairing, L is biotin and L′ is streptavidin. The L-O1 capture probe may contain one or more L moieties. For example, L-O1 may comprise 1, 2, 3, 4, 5, or more L moieties. In preferred aspects, L-O1 comprises one L moiety. A larger number of L moieties can be used to provide a higher capture affinity.

The signal probe X-O2 in these proposals includes at least one moiety X which is a signal moiety that provides an observable signal. The signal provided by X is detectable as evidence of the presence of the specific target bacteria. The signal may be provided either alone, in the absence of other components, or the moiety X may have an effect on contact with a further component to provide the detectable signal. The signal moiety X is not particularly limited and may be any known signal moiety. For example, it may be an enzyme, such as horseradish peroxidase (HRP) which catalyses the conversion of various substrates into another entity with an associated detectable change (such as a colour change). The moiety X may alternatively be selected from, radiolabels, fluorescent tags, europium, alkaline phosphatase, urease, β-galactasidase. In preferred embodiments X is HRP. In this case, the step of detecting the observable signal may comprise adding a chromogenic substrate that is acted upon by the HRP to convert it into a product with an associated colour change. The chromogenic substrates are known but may be selected from 3,3′,5,5′-Tetramethylbenzidine (TMB), 3,3′-Diaminobenzidine (DAB), 2,2′-azino-bis(3-ethylbenzothiazoline-6-sulphonic acid) (ABTS), and luminol. The X-O2 signal probe may contain one or more X moieties. For example, X-O1 may comprise 1, 2, 3, 4, 5, or more X moieties. In preferred aspects, X-O2 comprises one X moiety. A larger number of X moieties can be used to provide a higher detection signal from individual capture events.

The mode of detection of the detectable signal from the reaction depends on the nature of the signal provided by the moiety X. In preferred embodiments, the signal is a colour change or change in spectroscopic absorbance or emission of the reaction mixture. For example, a colour change or luminescence may, in the simplest cases, be detectable by visual inspection. In some cases a spectrometer may be used to detect the change in spectral behaviour of the reaction mixture.

In the capture probe L-O1 and the signal probe X-O2, the oligomers O1 and O2 are linked respectively to the L and X moieties by a linker unit (depicted as - herein). This linker unit for the signal probe is preferably as known and described in WO 2007/132207. This known linker for the signal probe is preferably 4,4′diisothiocyanato-2,2′-stilbenedisulfonic acid, disodium salt (DIDS). The linker L for the capture probe is preferably a polyethylene glycol (PEG) unit. Preferably the PEG unit has the structure H—(O—CH₂—CH₂)ₙ—OH in which n is an integer from 1 to 15. Preferably is an integer from 1-10, in particularly preferable embodiments, n=6.

The nature of the signal and capture probes leads to the ability to use whole-cell bacteria in the samples. It is thought that the relatively small size (e.g. less than 150 nucleotides for each of O1 and O2) of the probes and the relatively high rigidity of the linker between L and O1 and X and O2 both contribute to the beneficial ability of the probe moieties to penetrate whole-cell bacteria. For the signal probe, it is thought that the rigid link between the moiety X and O2 assists penetration of the larger X moiety into the bacteria without needing to previously extract the DNA or disrupt the cell wall. For the capture probe, it is thought that the small size of both the L and O1 units allow penetration of this probe into the bacteria relatively easily. This leads to an important benefit associated with the present methods, i.e. the ability to use whole-cell bacterial samples without DNA extraction steps. This means that the present methods are significantly simpler to perform than the existing analyses.

Furthermore, the simplicity and the nature of the probes mean that no hazardous reagents are required so the methods are typically safer than some known methods that employ hazardous reagents.

In some embodiments, the capture probe and signal probe mixture or the single linked capture/signal probe are provided in solid form. This solid form may become dissolved or suspended on addition of the sample or a reaction medium (e.g. water or buffer fluid). This solid form may be freeze dried. This is particularly relevant to the assay device described herein which includes a reagent chamber formed between two frangible or moveable barriers. In this device, the probes may be provided in solid (e.g. freeze dried) form between these frangible or moveable barriers.

In the present methods, the washing step is not particularly limited apart from the requirement to remove excess sample and probe composition. The washing may preferably be performed with a clean sample of the reaction solvent (e.g. water or a suitable reaction buffer).

The present proposals include a kit for the detection of specific whole-cell bacteria. The kit comprises the reagents and apparatus required to perform the detection which, in some preferred cases, may be a detection method as described herein.

The probe composition in the kit comprises a capture probe L-O1 and a signal probe X-O2 which are as defined herein.

The kit further includes an assay device which comprises a body including an assay chamber containing a substrate on which a moiety L′ is immobilised; and a sample collector/dispenser for collecting a sample to be assayed and dispensing a quantity of it into the assay chamber. L′ is as defined herein. The body and the collector/dispenser are engageable together.

The kit may further comprise a detector suitable for detecting the observable signal provided by the moiety X. For example the detector may be a spectrometer.

The present proposals further provide an assay device for use in a method of detection of specific whole-cell bacteria, e.g. a method as defined herein. The device comprises a body including an assay chamber and a reagent chamber, and a sample collector/dispenser for collecting a sample to be assayed and dispensing a quantity of it into the assay chamber. The body and the collector/dispenser are engageable together. In some cases this engagement is detachable, although in some embodiments it may be non-detachable. The reagent chamber is formed between two frangible or moveable barriers one of which separates the reagent chamber from the assay chamber. The reagent chamber contains a probe composition as defined herein.

The kits and assay devices described herein may be useful in methods according to the present proposals.

In an exemplary, albeit non-limiting, use of the assay device, a sample is collected in the sample collector/dispenser portion of the device. This sample collector/dispenser portion may, in some embodiments, be a syringe in which case a fluid sample may be drawn up into the syringe. The collector/dispenser portion is then engaged with the body of the device to dispense the sample into the assay chamber. In preferred embodiments, the action of engagement of the collector/dispenser portion with the body of the device breaks or displaces the frangible or moveable barriers between which the reagent chamber is defined. This breaking or displacement of the barriers allows the probe composition to pass from the reagent chamber into the assay chamber. The sample may then be dispensed into the reaction chamber which now contains the probe composition. The reaction mixture is allowed to incubate for a period of time (e.g. as defined herein) after which the mixture is extracted from the assay chamber (e.g. back into the collector/dispenser portion). The assay chamber is then washed, e.g. with clean reaction solvent (such as water or suitable reaction buffer) to remove excess sample and probe composition. In some cases the signal may then be detected directly. In some cases the signal detection step may further comprise the step of adding a further component as defined herein to provide the detectable signal (e.g. where the moiety X is an enzyme, the detection step may comprise addition of an enzyme substrate on which the enzyme acts to effect a detectable change, such as a colour change).

In some cases the general structure of the assay device may be a known assay device such as that defined in WO 93/09431. Such a device may be as sold under the trade name SafeTube®. In some cases the assay device may be modified from that defined in WO 93/09431 by removal of the non-detachable engagement between the collector/dispenser portion and the body of the device so that these portions can be detached after engagement.

As usual, any of the features associated with the methods, apparatus, kits, or uses defined herein are independent and may be freely combined with other features of the methods, apparatus, kits, or uses as appropriate and where they are described in the same context. Features described in relation to the proposed methods may also be applicable to the uses. Features of the components used in the proposed methods are also generally applicable to the apparatus, kits, and uses.

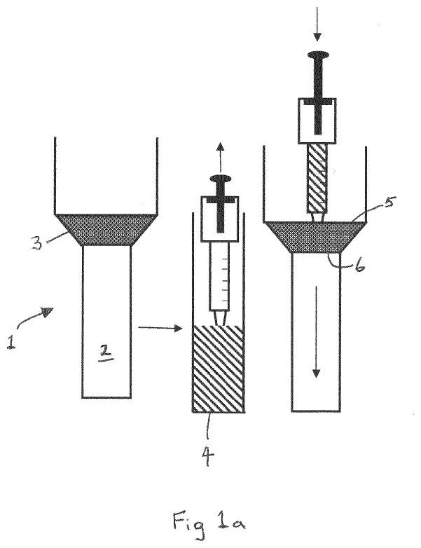

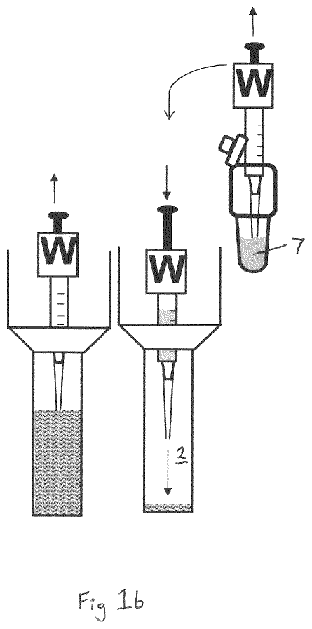

FIGS. 1a-c show an exemplary assay device (SafeTube C)) and its use in an assay. FIG. 1a shows the “target” step. The device 1 comprises an assay chamber 2 coated with one half of the binding pair L/L′ (e.g. streptavidin) and a reagent chamber 3 which contains freeze dried capture and signal probes (e.g. capture oligomer bound via PEG group to biotin at the 3′ end and signal oligomer bound via 4,4′diisothiocyanato-2,2′-stilbenedisulfonic acid, disodium salt (DIDS) to horseradish peroxidase at the 5′ end).

A portion (e.g. 200 μl) of a test solution for analysis 4 is drawn up into the syringe T. The syringe T is inserted through two barriers 5/6 to allow the freeze dried mixture of signal and capture probes to fall into the assay chamber 2. The test solution 4 is then discharged into the assay chamber 2 to dissolve the freeze dried probe mix. The assay chamber is then inserted into a colourimeter to record a background reading.

FIG. 1b shows a wash step in which a wash solution 7 is drawn up into a syringe W and subsequently discharged into the assay chamber 2. Following agitation, the wash solution 7 is withdrawn from the assay chamber 2 using the same syringe W. The wash procedure is typically repeated (a total of two times).

FIG. 1c shows a colour development step in which a solution of enzyme colour developer 8 is drawn up into a syringe C. The colour developer is selected according to the signal moiety X and interacts with the moiety X to produce a colour change (e.g. 3,3′,5,5′-tetramethylbenzidine (TMB) to interact with horseradish peroxidase). The colour developer 8 is discharged into the assay chamber 2 and allowed to incubate to develop any colour change. The assay chamber 2 is then inserted into a colourimeter to records any colour change.

### EXAMPLES

The following examples are not intended to limit the scope of the claims; they are provided as exemplary embodiments.

### Example 1

This example aims to test a developed assay that aims to objectively quantify antibiotic resistant bacterial infections within patient samples taken from a hospital setting. This is to screen for carriers of antibiotic resistant MRSA. MRSA represent Staphylococcus aureus bacterial infections that carry antibiotic resistance and prove significant and numerous enough risk within present healthcare, representing a unanimous growing concern prompting mitigation strategies.

Genomic DNA positive target represents DNA harvested from MRSA bacteria that carry the meca gene corresponding to antibiotic resistance. In order to validate the quantified data a negative control MSSA genomic DNA was run alongside, thereby representing Staphylococcus aureus infections that do not contain any meca gene providing a negative control for comparison against positive samples. This established confirmation of detection of cultured whole cell MRSA and differentiated negative MSSA cultures of various strains.

Testing was performed using the general protocol outlined in FIG. 1a-c.

Single use SafeTube® apparatus were prepared comprising Streptavidin coated tubes with freeze dried probe reagent. The tubes were loaded with 120 pmol Streptavidin. In the reagent chamber of the SafeTube® apparatus, was provided a duel probe mix (Biotin labelled capture oligomer and HRP-labelled signal oligomer).

The capture probe comprises a capture oligomer sequence of SEQ ID NO: 1:

3′-gaa atg cta ttt ttc gag gtt gta ctt cta-5′

With biotin attached via a hexaethylene glycol linker at the 3′ end of the oligomer.

The signal probe comprises a signal oligomer sequence of SEQ ID NO: 2:

3′-ag tgt tag caa ctg cta tta tcg tta tgt t-5′

With horseradish peroxidase (HRP) attached via a 4,4′diisothiocyanato-2,2′-stilbenedisulfonic acid, disodium salt (DIDS) linker at the 5′ end of the oligomer.

### Test Protocol

The test protocol below was established for every MRSAScreen test carried out ensuring continuity throughout the experimental process.

MRSA SafeTube described above including Streptavidin coated tube with probe disc containing assay reagents was unpacked.

An aliquot of 200 μl deionised water was punctured through the disc to flush the reagent mixture down to the bottom of the tube.

Genomic DNA was supplied by Northampton University to emulate whole cell bacterial samples, positive and negative samples as shown below.
Positive genomic MRSA target MU50
Negative genomic MSSA target MSSA 110

Genomic DNA (Target) was added via Gilson pipette at the bottom of the tube at required concentration, 30 ng for this set of tests.

The mixture was agitated for 5 seconds and allowed to stand for an incubation period of 15 minutes. This is to allow for the probes and target to mix and form complexes that will adhere to streptavidin if positive target is present.

Wash procedure was performed using 1 ml wash buffer (0.1% Tween in PBS) and the probe mix and unbound genomic was evacuated. This wash procedure was repeated twice.

Enzyme Peroxidase substrate colour development 3,3′,5,5′-tetramethylbenzidine (TMB) 500 μl was added via syringe which then locks down the system enclosing the test in the SafeTube®. In positive samples the colour change is from colourless to blue and occurs rapidly with a peak colour developing after about 10 minutes. With a negative result, small amounts of blue colour are expected due to the sensitivity of the assay. Typically, positive and negative results can be reliably differentiated after 1-2 minutes.

The test was then inserted immediately into a colourimeter instrument (Micro stepping motor with integrating sphere colourimeter for logging optical transmittance of colour solutions at 655 nm as supplied by Bentham instruments) and data logging commenced.

Data was logged for 5 minutes onto laptop with positive and negative readings being noted for statistical analysis.

### Results

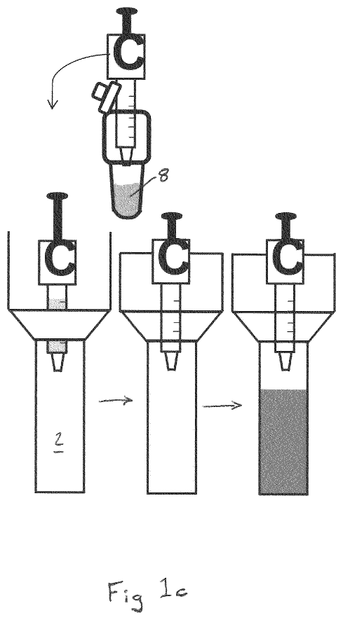

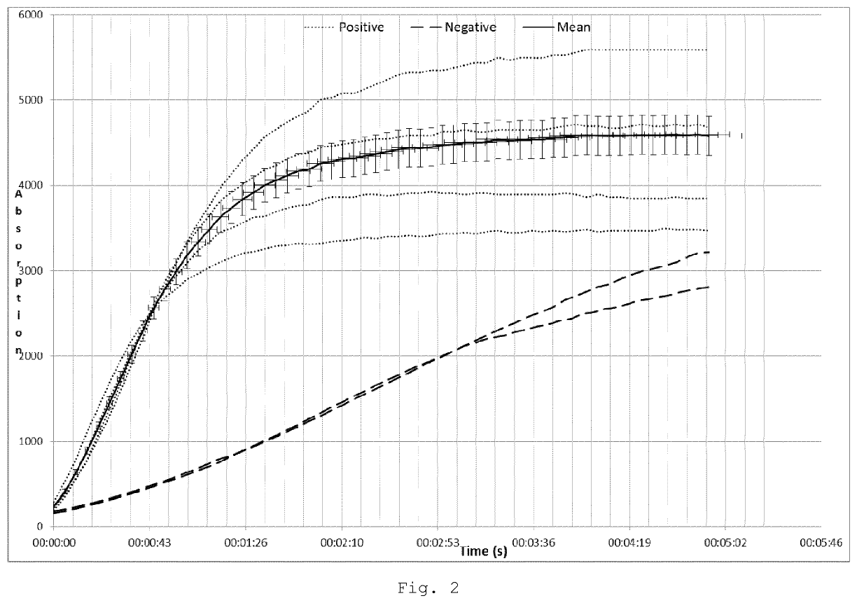

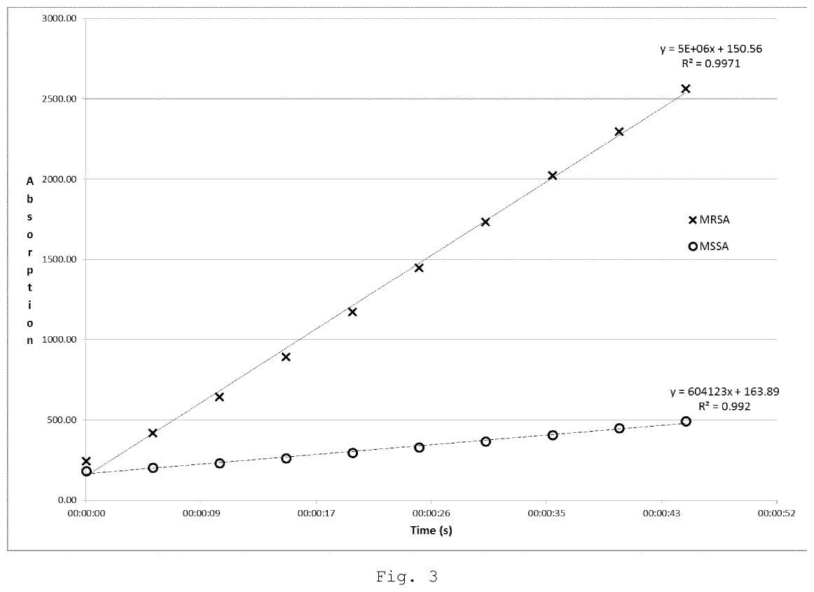

Results are shown in the graph in FIG. 2 and FIG. 3 represents a comparison of the negative and positive data that was produced. FIG. 3 is an enlargement of a portion of FIG. 2. Positive data (dotted line), with mean shown as a solid line, represents the positive MRSA genomic results with mean peak absorption around 4700 upon termination. Negative MSSA data has been plotted with dashed trend line with peak absorption around 3000 at termination.

These two regressions are compared to one another in order to determine if they are statistically significantly different. As demonstrated FIG. 2, MRSA positive (dotted) fit steeper positive linear regressions models compared to the negative MSSA results (dashed lines). With a p value of 0.000 we can therefore determine that a significant difference exists between the positive and negative tests when the termination point around the one minute mark. We therefore reject the null hypnosis and accept our alternate hypnosis thereby demonstrating that via quantification we are able to successfully demonstrate a difference between positive MRSA results and negative MSSA results using the MRSAScreen test.

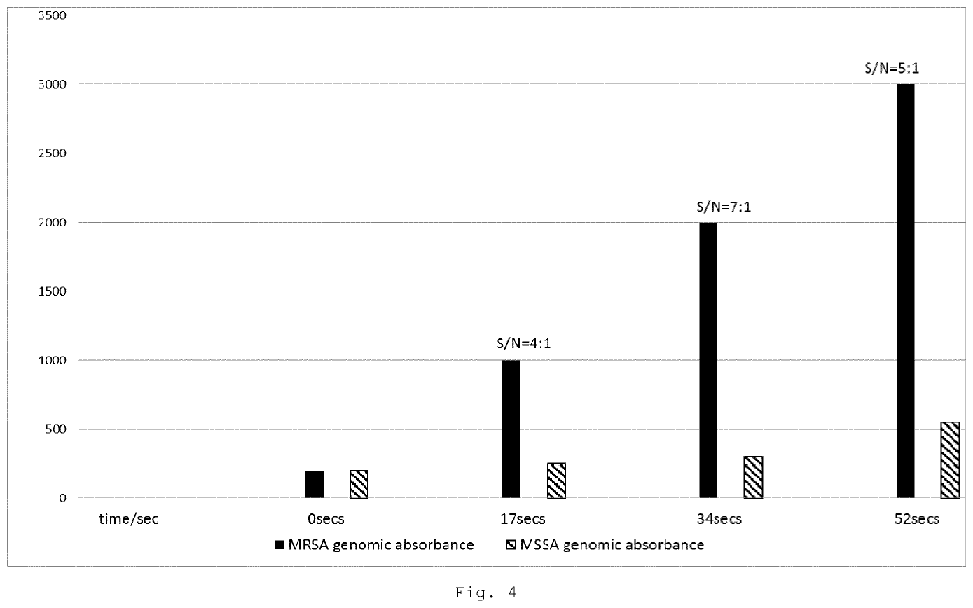

FIG. 4 shows a plot of absorbance of the MRSA and MSSA samples at various selected time points showing the rapid development of the higher absorbance for MRSA as compared to MSSA which results in the high sensitivity and rapid detection capabilities of the present test. As a comparison, the S/N ratio for 20 ng genomic mycobacterium immunogen and Factor V in a known microtitre well assay is approximately 2:1 at a detection time of about 5 mins.

### Example 2

Whole cell bacterial samples of various strains where grown up via the culture technique with growth media. All starting cultures were prepared to an optical density of 0.1±0.01. Starting cultures were then enumerated and dilutions made to determine lower detection limits.

Testing was performed using the protocol described above in Example 1.

Data was gathered using a standard Spectrophotometer with readings being obtained by adding 250 μl PBS to the 500 μl TMB reagent (after the test had been completed). This was so that the spectrophotometer had enough liquid in the cuvette to obtain a reading.

Detection limits where determined as being negative when number of cells were so low that no disenable difference from MSSA samples could be made i.e. when absorbance was under 0.1 after 30 seconds.

Table 1 below shows the range of strains that were tested.

TABLE 1

| | | Lower | |
|---|---|---|---|
| | Starting | detection | |
| | number of | limit | |
| | cells (OD | (number of | |
| Strain | 0.1 ± 0.01) | cells/ml) | Result |
| MRSA mw2 | 2.4 × 10⁶ | 3.4 | Positive |
| MRSA pvl ca | 1.5 × 10⁶ | 15 | Positive |
| MRSA 11 | 4.6 × 10⁷ | 4.6 | Positive |
| MRSA mu50 | 7.8 × 10⁸ | 78 | Positive |
| MRSA 13142 | 2.8 × 10⁸ | 28 | Positive |
| MSSA 13297 | 2 × 10⁶ | 2 × 10⁶ | Negative |
| MSSA 110 | 4.7 × 10⁷ | 4.7 × 10⁷ | Negative |

This consists of 5 positive MRSA strains and 2 variations of MSSA negative. The table clearly demonstrates meaningful differential between positive and negative readings with strong continuity, i.e. all positives were positive towards a lower detection limit that is many time smaller that the negative limits found. This also demonstrates that all negative MSSA strains were confirmed negative using this test with no false negative results. It can therefore be concluded that a clear demonstration MRSAScreen was shown to work on whole cell MRSA cultured samples to very high levels of sensitivity, only a few cells per ml.

### Example 3

Following the protocol of Example 2 further whole cell samples were prepared using Mycobacterium Immunogen in an oil medium. These samples were prepared with very low levels of bacteria; all starting concentrations were 10³ cells/ml. A control sample was also prepared using the same medium but without the bacteria.

Analysis was performed as set out in Example 2 using Biotin labelled capture oligomer and HRP-labelled signal oligomer with the oligomers specific for mycobacterium immunogen. However in this Example the studies were performed in streptavidin-coated microtitre wells rather than the streptavidin-coated SafeTube® used in Example 2.

Spectrophotometer data was gathered as set out in Example 2 and the results are presented in Table 2. For each experimental run, the absorbance at the lower detection limit (10 cells/ml) was recorded and is also shown in Table 2.

TABLE 2

| | | | Absorbance | |
|---|---|---|---|---|
| | Starting | | at 10 | |
| | number of | Lower | cells/ml | |
| | cells | detection | (lower | |
| Run | (number of | limit (number | detection | |
| Number | cells/ml) | of cells/ml) | limit) | Result |
| Run 1 | 10⁵ | 10 | 1.15 | Positive |
| Run 2 | 10⁵ | 10 | 1.18 | Positive |
| Run 3 | 10⁵ | 10 | 1.29 | Positive |
| Control | 0 | — | 0.4 | Negative |

1. A method of detecting specific whole-cell bacteria, the method comprising:
adding a probe composition to a sample comprising whole bacterial cells, the probe composition comprising a capture probe L-O1 and a signal probe X-O2, wherein L is at least one moiety that is one half of a binding pair, X is at least one signal moiety that provides an observable signal, and each O1 and O2 is independently a 15-150 nucleotide oligomer that is complementary to a portion of a nucleotide sequence of the specific bacteria;
bringing the sample/probe composition mixture into contact with a substrate on which a moiety L′ is immobilised wherein L′ is complementary to and binds with the moiety L;
washing the substrate to remove excess sample and probe composition; and
detecting the observable signal.

2. A method according to claim 1, wherein the specific whole-cell bacteria is selected from Bacillus anthracis (anthrax), Yersinia pestis (plague), methicillin-resistant Staphylococcus aureus (MRSA), methicillin-susceptible Staphylococcus aureus (MSSA), Clostridium difficile (C. diff), Vancomycin-resistant Enterococci (VRE), Carbapenemase-producing enterobacteriaceae (CPE), Multidrug resistant Mycobacterium tuberculosis (MDR-TB), colistin resistant bacteria, Extended-spectrum β-lactamase producing Gram-negative bacteria (ESBLs), Klebsiella pneumoniae carbapenemase (KPC) producing Gram-negative bacteria, Streptococcus pneumoniae, Haemophilus influenza, Mycobacterium tuberculosis, Shigellae (e.g. S. dysenteriae, S. flexneri, S. boydii, S. sonnei), Campylobacter, Salmonellae (e.g. Salmonella enterica, Salmonella bongori, Salmonella Typhi), Escherichia coli, Lysteria monocytogenes, Shewanella oneidensis, Neisseria meningitides, Treponema pallidum (syphilis), Neisseria gonorrhoeae (gonorrhoea), Chlamydia trachomatis (chlamydia), Porphyromonas gingivalis.

3. A method according to claim 1, wherein the specific whole-cell bacteria is selected from drug resistant bacteria.

4. A method according to claim 3, wherein the drug resistant bacteria is selected from methicillin-resistant Staphylococcus aureus (MRSA), Carbapenemase-producing enterobacteriaceae (CPE), Multidrug resistant Mycobacterium tuberculosis (MDR-TB), and colistin resistant bacteria.

5. A method according to claim 1, wherein the capture probe comprises a capture oligomer sequence O1:
3′-gaa atg cta ttt ttc gag gtt gta ctt cta-5′
and/or the signal probe comprises a signal oligomer sequence O2:
3′-ag tgt tag caa ctg cta tta tcg tta tgt t-5′,

6. A method according to claim 1, wherein in the capture probe L-O1, L is biotin attached to O1 via a PEG linker; and/or wherein in the signal probe X-O2, X is horseradish peroxidase (HRP) attached to O2 via a 4,4′ diisothiocyanato-2,2′-stilbenedisulfonic acid, disodium salt (DIDS) linker.

7. A kit for the detection of specific whole-cell bacteria, the kit comprising:
a probe composition comprising a capture probe L-O1 and a signal probe X-O2, wherein L is at least one moiety that is one half of a binding pair, X is at least one signal moiety that provides an observable signal, and each O1 and O2 is independently a 15-150 nucleotide oligomer that is complementary to a portion of a nucleotide sequence of the specific bacteria; and
an assay device comprising: a body including an assay chamber containing a substrate on which a moiety L′ is immobilised wherein L′ is complementary to and binds with the moiety L; a sample collector/dispenser for collecting a sample to be assayed and dispensing a quantity of it into the assay chamber, wherein the body and the collector/dispenser are engageable together.

8. An assay device for use in a method of detection of specific whole-cell bacteria comprising: a body including an assay chamber and a reagent chamber, and a sample collector/dispenser for collecting a sample to be assayed and dispensing a quantity of it into the assay chamber,
the body and the collector/dispenser being engageable together,
the reagent chamber is formed between two frangible or moveable barriers one of which separates the reagent chamber from the assay chamber,
wherein the reagent chamber contains a probe composition comprising a capture probe L-O1 and a signal probe X-O2, wherein L is a moiety that is one half of a binding pair, X is a signal moiety that provides an observable signal, and each O1 and O2 is independently a 5-100 nucleotide oligomer that is complementary to a portion of a nucleotide sequence of the specific bacteria, and the assay chamber contains a substrate on which a moiety L′ is immobilised wherein L′ is complementary to and binds with the moiety L.

9. A kit according to claim 7, wherein the specific whole-cell bacteria is selected from Bacillus anthracis (anthrax), Yersinia pestis (plague), methicillin-resistant Staphylococcus aureus (MRSA), methicillin-susceptible Staphylococcus aureus (MSSA), Clostridium difficile (C. diff), Vancomycin-resistant Enterococci (VRE), Carbapenemase-producing enterobacteriaceae (CPE), Multidrug resistant Mycobacterium tuberculosis (MDR-TB), colistin resistant bacteria, Extended-spectrum β-lactamase producing Gram-negative bacteria (ESBLs), Klebsiella pneumoniae carbapenemase (KPC) producing Gram-negative bacteria, Streptococcus pneumoniae, Haemophilus influenza, Mycobacterium tuberculosis, Shigellae (e.g. S. dysenteriae, S. flexneri, S. boydii, S. sonnei), Campylobacter, Salmonellae (e.g. Salmonella enterica, Salmonella bongori, Salmonella Typhi), Escherichia coli, Lysteria monocytogenes, Shewanella oneidensis, Neisseria meningitides, Treponema pallidum (syphilis), Neisseria gonorrhoeae (gonorrhoea), Chlamydia trachomatis (chlamydia), Porphyromonas gingivalis.

10. A kit according to claim 7, wherein the specific whole-cell bacteria is selected from drug resistant bacteria.

11. A kit or assay device according to claim 10, wherein the drug resistant bacteria is selected from methicillin-resistant Staphylococcus aureus (MRSA), Carbapenemase-producing enterobacteriaceae (CPE), Multidrug resistant Mycobacterium tuberculosis (MDR-TB), and colistin resistant bacteria.

12. A kit according to claim 7, wherein the capture probe comprises a capture oligomer sequence O1:
3′-gaa atg cta ttt ttc gag gtt gta ctt cta-5′
and/or the signal probe comprises a signal oligomer sequence O2:
3′-ag tgt tag caa ctg eta tta tcg tta tgt t-5′,

13. A kit according to claim 7, wherein in the capture probe L-O1, L is biotin attached to O1 via a PEG linker; and/or wherein in the signal probe X-O2, X is horseradish peroxidase (HRP) attached to O2 via a 4,4′diisothiocyanato-2,2′-stilbenedisulfonic acid, disodium salt (DIDS) linker.

14. A kit according to claim 13, wherein L′ is streptavidin.

15. (canceled)

16. An assay device according to claim 8, wherein the specific whole-cell bacteria is selected from Bacillus anthracis (anthrax), Yersinia pestis (plague), methicillin-resistant Staphylococcus aureus (MRSA), methicillin-susceptible Staphylococcus aureus (MSSA), Clostridium difficile (C. diff), Vancomycin-resistant Enterococci (VRE), Carbapenemase-producing enterobacteriaceae (CPE), Multidrug resistant Mycobacterium tuberculosis (MDR-TB), colistin resistant bacteria, Extended-spectrum β-lactamase producing Gram-negative bacteria (ESBLs), Klebsiella pneumoniae carbapenemase (KPC) producing Gram-negative bacteria, Streptococcus pneumoniae, Haemophilus influenza, Mycobacterium tuberculosis, Shigellae (e.g. S. dysenteriae, S. flexneri, S. boydii, S. sonnei), Campylobacter, Salmonellae (e.g. Salmonella enterica, Salmonella bongori, Salmonella Typhi), Escherichia coli, Lysteria monocytogenes, Shewanella oneidensis, Neisseria meningitides, Treponema pallidum (syphilis), Neisseria gonorrhoeae (gonorrhoea), Chlamydia trachomatis (chlamydia), Porphyromonas gingivalis.

17. An assay device according to claim 8, wherein the specific whole-cell bacteria is selected from drug resistant bacteria.

18. An assay device according to claim 17, wherein the drug resistant bacteria is selected from methicillin-resistant Staphylococcus aureus (MRSA), Carbapenemase-producing enterobacteriaceae (CPE), Multidrug resistant Mycobacterium tuberculosis (MDR-TB), and colistin resistant bacteria.

19. An assay device according to claim 8, wherein the capture probe comprises a capture oligomer sequence O1:
3′-gaa atg cta ttt ttc gag gtt gta ctt cta-5′
and/or the signal probe comprises a signal oligomer sequence O2:
3′-ag tgt tag caa ctg eta tta tcg tta tgt t-5′.

20. An assay device according to claim 8, wherein in the capture probe L-O1, L is biotin attached to O1 via a PEG linker; and/or wherein in the signal probe X-O2, X is horseradish peroxidase (HRP) attached to O2 via a 4,4′diisothiocyanato-2,2′-stilbenedisulfonic acid, disodium salt (DIDS) linker.

21. An assay device according to claim 20, wherein L′ is streptavidin.
